(19) United States
(12) Patent Application Publication
(10) Pub. No.: US 2025/0186415 A1
(43) Pub. Date: Jun. 12, 2025

(54) EXTENDED-RELEASE PHARMACEUTICAL COMPOSITIONS FOR TREATING EYE CONDITIONS

(71) Applicant: Harrow IP, LLC, Nashville, TN (US)

(72) Inventors: Dennis E. Saadeh, Nashville, TN (US); Mark L. Baum, Nashville, TN (US); Richard L. Lindstrom, Wayzata, MN (US)

(21) Appl. No.: 18/844,789

(22) PCT Filed: Mar. 7, 2022
(86) PCT No.: PCT/US2022/019167

§ 371 Date: Sep. 6, 2024

(51) Int. Cl. A61K 31/439; A61K 9/00; A61K 9/08; A61K 31/40; A61K 31/46; A61K 31/5386; A61K 47/02; A61K 47/10; A61K 47/18; A61K 47/32; A61K 47/36

(52) U.S. Cl. CPC A61K 31/439; A61K 9/0019; A61K 9/08; A61K 31/40; A61K 31/46; A61K 31/5386; A61K 47/02; A61K 47/10; A61K 47/183; A61K 47/32; A61K 47/36

(57) ABSTRACT

The present disclosure encompasses methods and compositions for extended-release formulations for treating ophthalmic conditions. These formulations comprise a pharmaceutical agent, a cationic polymer, a non-ionic polymer, and a pharmaceutical carrier and are essentially preservative free. The formulations provided herein are particularly useful for sustained and controlled release of drugs and have low side effects.

### FIELD OF THE DISCLOSURE

The present disclosure relates generally to the field of pharmaceuticals and more specifically to sustained release ophthalmic pharmaceutical compositions with low levels of preservatives and high stability.

### BACKGROUND

Ophthalmic medications usually rely on topical application of the medication on the cornea or outer edges of the eye. A good ophthalmic drug needs to have several desirable characteristics including good corneal penetration, high residence time, low susceptibility to leaks and loss during application. Currently available topical applications usually require multiple daily applications to account for leakage and low penetration rates. In addition, these drugs are formulated with preservatives and have undesirable side-effects like redness, itching, tearing and stickiness. The need for multiple applications reduces compliance while enhancing side effects due to repeated exposure to preservatives.

What is needed are safe, slow release, low preservative, high stability pharmaceutical formulations for eye conditions and diseases.

### SUMMARY OF THE DISCLOSURE

Described herein are sterile pharmaceutical composition for contacting an ocular surface of a mammal, the sterile pharmaceutical composition comprising: a pharmaceutical agent, a cationic polymer, a non-ionic polymer, and a pharmaceutical carrier, wherein the duration of the clinical effect stemming from the sterile pharmaceutical composition is greater than 6 hours.

In some aspects the sterile pharmaceutical composition disclosed herein comprise a pharmaceutical agent for the treatment of ophthalmic diseases and conditions including but not limited to presbyopia, myopia, droopy or red eyes.

In some aspects the sterile pharmaceutical composition further comprising an anionic polymer. In some exemplary aspects the anionic polymer may comprise polycarbophil, carbomer and combinations thereof. In some aspects the anionic polymer has a concentration of about 0.2% w/v to about 2.0% w/v.

In some aspects of the current disclosure, the cationic polymer in the sterile pharmaceutical composition has a concentration of about 0.01% w/v to about 2.0% w/v.

In some aspects the sterile pharmaceutical composition further comprises one or more additional non-ionic polymers for example polyvinyl pyrrolidone (Povidine K30), a poly (oxyethylene-co-oxypropylene) block copolymer (Poloxamer 407), or combination thereof at a concentration of about 0.1% w/v to about 4.0% w/v.

In some aspects the pharmaceutical carrier in the sterile pharmaceutical composition comprises one or more pharmaceutically acceptable buffers. Some examples of pharmaceutically acceptable buffers include but are not restricted to phosphate buffer, bicarbonate, carbonate buffer, succinate buffer, borate buffer, cacodylate buffer, citrate buffer, a zwitterionic buffer, a tris (hydroxymethyl) aminomethane (TRIS) buffer, morpholine propanesulphonic acid (MOPS), N-(2-hydroxyethyl) piperazine-N′ (2-ethanesulfonic acid) (HEPES), an acetate buffer and any combination thereof.

In some aspects the sterile pharmaceutical composition comprises one or more tonicity adjusting agent examples of which include but are not restricted glycerin, sodium chloride, potassium chloride, magnesium chloride, calcium chloride, and any combination thereof.

In some aspects the sterile pharmaceutical composition comprises pharmaceutical carriers comprising one or more stabilizers examples of which include but are not restricted to EDTA, sodium thiosulfate, sodium metabisulfite and any combinations thereof.

In some aspects the sterile pharmaceutical composition comprises the pharmaceutical carrier comprises one or more pH adjusting agents and has a pH between 5.0 to about 8.0.

In some aspects the sterile pharmaceutical composition comprises a pharmaceutical carrier comprising water.

In some aspects the pharmaceutical composition does not comprise a preservative and wherein the pharmaceutical composition has a viscosity of about 1 cP to about 20 cP.

In some exemplary aspects, the pharmaceutical agent comprises a cholinesterase inhibitor examples of which include but are not restricted to physostigmine, neostigmine, pyridostigmine, ambenonium, demecarium bromide, rivastigmine, galantamine, caffeine, rosmarinic acid, alpha-pinene, donepezil, tacrine, edrophonium, huperzine A, ladostigil, ungeremine, lactucopicrin, echothiophate, diisopropyl fluorophosphatesneostigmine, echothiophate iodide (also known as phospholine iodide), aceclidine, ambenonium, demecarium, rivastigmine, galantamine, acotiamide, diisopropyl fluorophsphate, cadusafos, chlorpyrifos, cyclosarin, dichlorvos, dimethoate, metrifonate, and any combination or pharmaceutically acceptable salts thereof. In some aspects the cholinesterase inhibitor has a concentration of about 0.001% w/v to about 0.1% w/v.

In some exemplary aspects the pharmaceutical agent comprises a muscarinic receptor agonist for instance pilocarpine, choline, acetylcholine, nicotine, methacholine, carbachol, cevimeline, CI-1017, bethanechol, milameline, muscarine, oxotremorine, sabcomeline, talsaclidine, tazomeline, vedaclidine, VU0152100, VU0238429, xenomeline, AF102B, AF150(S), AF267B, aceclidine, arecoline, pilocarpine, cevimeline, and any combination or pharmaceutically acceptable salts thereof. In some aspects the muscarinic agonist has a concentration of about 0.001% w/v to about 1% w/v.

In some aspects the pharmaceutical agent comprises a muscarinic receptor antagonist examples of which include but are not restricted to atropine, scopolamine, glycopyrrolate, ipratropium bromide and any combination thereof or any pharmaceutically acceptable salt thereof at a concentration of about 0.001% w/v to about 3% w/v.

In some aspects the pharmaceutical composition has a tropic acid concentration of less than 0.01 mg/mL after 180 days of storage in a sealed container at a temperature of 25° C. (+2) and 60% (+5%) relative humidity and is stable and potent for 60 days, 120 days, or 180 days of storage in a sealed container at a temperature of 25° C. (+2) and 60% (+5%) relative humidity.

In some aspects, the pharmaceutical composition is packaged in multi-dose bottles.

In some aspects the pharmaceutical composition does not cause burning, stinging, tearing, or hyperemia.

In some aspects the pharmaceutical agent in the steril pharmaceutical composition comprises an alpha-adrenergic agonist, examples of which include phenoxybenzamine, phentolamine, tolazoline, trazodone, alfuzosin, dapiprazole, thymoxamine, doxazosin, prazosin, tamsulosin, bunezosin, terazosin, trimazosin, silodosin, atipamezole, idazoxan, mirtazapine, yohimbine, carvedilol, labetalol, urapidil, abanoquil, adimolol, ajmalicine, amosulalol, arotinolol, atiprosin, benoxathian, buflomedil, bunazosin, carvedilol, CI-926, corynanthine, DL-017, domesticine, eugenodilol, fenspiride, GYM-12743, GYKI-16084, indoramin, ketanserin, L-765314, mephendioxan, metazosin, monatepil, naftopidil, nantenine, neldazosin, nicergoline, niguldipine, pelanserin, phendioxan, piperoxan, quinazosin, ritanserin, RS-97078, SGB-1534, SL-890591, spiperone, talipexole, tibalosin, tiodazosin, tipentosin, tolazoline, upidosin, zolertine, a pharmaceutically acceptable salt thereof, and combinations thereof, and any combination or pharmaceutically-acceptable salts thereof.

In some aspects the current disclosure also encompasses packaged sterile pharmaceutical composition for contacting an ocular surface of a mammal, said sterile pharmaceutical composition comprising: a pharmaceutical agent, a cationic polymer, and a non-ionic polymer, wherein the duration of the clinical effect stemming from the sterile pharmaceutical composition is greater than 6 hours, wherein the sterile pharmaceutical composition does not comprise a preservative, and wherein the composition is packaged in a multi-dose bottle.

In some exemplary aspects of the current disclosure the packaged sterile pharmaceutical composition comprises pharmaceutical agent for the treatment of presbyopia, myopia, droopy or red eyes.

In some aspects the packaged sterile pharmaceutical composition may comprise one or more additional non-ionic polymers for example polyvinyl pyrrolidone (povidone K30), a poly (oxyethylene-co-oxypropylene) block copolymer (poloxamer 407), or combinations thereof at a concentration of about 0.1% w/v to about 4.0% w/v.

In some aspects the packaged sterile pharmaceutical composition may comprise an anionic polymer at a concentration of about 0.2% w/v to about 2.0% w/v.

In some aspects the packaged sterile pharmaceutical composition may comprise a cationic polymer like chitosan at concentration of about 0.01% w/v to about 2.0% w/v.

In some aspects the packaged sterile pharmaceutical composition may comprise a pharmaceutical carrier comprising one or more buffers, tonicity adjusting agents, stabilizers, water etc and has a viscosity of about 1 cP to about 20 cP and has a pH of about 5.0 to about 8.0.

In some aspects the packaged sterile pharmaceutical composition may comprises a cholinesterase inhibitor for example physostigmine, neostigmine, pyridostigmine, ambenonium, demecarium bromide, rivastigmine, galantamine, caffeine, rosmarinic acid, alpha-pinene, donepezil, tacrine, edrophonium, huperzine A, ladostigil, ungeremine, lactucopicrin, echothiophate, diisopropyl fluorophosphatesneostigmine, echothiophate iodide (also known as phospholine iodide), aceclidine, ambenonium, demecarium, rivastigmine, galantamine, acotiamide, diisopropyl fluorophsphate, cadusafos, chlorpyrifos, cyclosarin, dichlorvos, dimethoate, metrifonate, and any combination or pharmaceutically acceptable salts thereof at a concentration of about 0.001% w/v to about 0.1% w/v.

In some aspects the packaged sterile pharmaceutical composition may comprises a muscarinic receptor agonist for example pilocarpine, choline, acetylcholine, nicotine, methacholine, carbachol, cevimeline, CI-1017, bethanechol, milameline, muscarine, oxotremorine, sabcomeline, talsaclidine, tazomeline, vedaclidine, VU0152100, VU0238429, xenomeline, AF102B, AF150(S), AF267B, aceclidine, arecoline, pilocarpine, cevimeline, and any combination or pharmaceutically acceptable salts thereof at a concentration of about 0.001% w/v to about 1.5% w/v.

In some aspects the packaged sterile pharmaceutical composition comprises a muscarinic receptor antagonist for example atropine, scopolamine, glycopyrrolate, ipratropium bromide and any combination thereof or any pharmaceutically acceptable salt thereof. In some aspects the atropine has a concentration of about 0.001% w/v to about 3% w/v.

In some aspects the packaged sterile pharmaceutical composition has a tropic acid concentration of less than 0.01 mg/mL after 180 days of storage in a sealed container at a temperature of 25° C. (±2) and 60% (±5%) relative humidity and is stable for at least 60 days, 120 days, or 180 days of storage.

In some aspects the packaged sterile pharmaceutical composition does not cause burning, tearing, hyperemia or stinging.

In some aspects the packaged sterile pharmaceutical composition comprises an alpha-adrenergic agonist for example apraclondine, brimonidine, oxymetazoline, and any combinations thereof and any pharmaceutically acceptable salts thereof.

In some aspects the current disclosure also encompasses packaged sterile pharmaceutical composition for contacting an ocular surface of a mammal for the treatment of myopia, the sterile pharmaceutical composition comprising: a pharmaceutical agent, a cationic polymer, and a non-ionic polymer, wherein the duration of the clinical effect stemming from the sterile pharmaceutical composition is greater than 6 hours.

In some aspects the packaged sterile pharmaceutical composition does not comprise a preservative.

In some aspects the packaged sterile pharmaceutical composition is packaged in multi-dose bottles.

In some aspects the packaged sterile pharmaceutical composition does not cause burning tearing, or stinging.

The packaged sterile pharmaceutical composition of claim 71, wherein the pharmaceutical agent comprises atropine.

In some aspects the packaged sterile pharmaceutical composition has a stable pH (±0.5% of starting pH) for at least 180 days of storage in a sealed container at a temperature of 25° C. (±2) and 60% (±5%) relative humidity and remains potent comprising ±10% of the pharmaceutical agent's concentration for at least 6 months of storage.

In some aspects the current disclosure also encompasses sterile pharmaceutical composition for contacting an ocular surface of a mammal for the treatment of presbyopia, the sterile pharmaceutical composition comprising: a pharmaceutical agent, a cationic polymer, and a non-ionic polymer, wherein application of the composition results in a pupil diameter of no more than 2 mm for at least 6 hours. In some aspects the sterile pharmaceutical composition does not comprise a preservative and may be packaged in multi-dose bottles.

In some aspects the sterile pharmaceutical composition for contacting an ocular surface of a mammal for the treatment of presbyopia comprises a cholinesterase inhibitor for example physostigmine, neostigmine, pyridostigmine, ambenonium, demecarium bromide, rivastigmine, galantamine, caffeine, rosmarinic acid, alpha-pinene, donepezil, tacrine, edrophonium, huperzine A, ladostigil, ungeremine, lactucopicrin, echothiophate, diisopropyl fluorophosphatesneostigmine, echothiophate iodide (also known as phospholine iodide), aceclidine, ambenonium, demecarium, rivastigmine, galantamine, acotiamide, diisopropyl fluorophsphate, cadusafos, chlorpyrifos, cyclosarin, dichlorvos, dimethoate, metrifonate, and any combination or pharmaceutically acceptable salts thereof.

In some aspects the sterile pharmaceutical composition for contacting an ocular surface of a mammal for the treatment of presbyopia comprises an alpha-adrenergic agonist for example apraclonidine, brimonidine, oxymetazoline, and any combinations thereof, and any pharmaceutically acceptable salt thereof.

In some aspects the sterile pharmaceutical composition for contacting an ocular surface of a mammal for the treatment of presbyopia comprises a muscarinic receptor agonist for examplepilocarpine, choline, acetylcholine, nicotine, methacholine, carbachol, cevimeline, CI-1017, bethanechol, milameline, muscarine, oxotremorine, sabcomeline, talsaclidine, tazomeline, vedaclidine, VU0152100, VU0238429, xenomeline, AF102B, AF150(S), AF267B, aceclidine, arecoline, pilocarpine, cevimeline, and any combination or pharmaceutically acceptable salts thereof at a concentration of about 0.001% w/v to about 1.5% w/v.

In some aspects the sterile pharmaceutical composition for contacting an ocular surface of a mammal for the treatment of presbyopia comprises muscarinic receptor antagonist for example atropine, scopolamine, glycopyrrolate, ipratropium bromide and any combination thereof or any pharmaceutically acceptable salt thereof. In some aspects the atropine has a concentration of about 0.001% w/v to about 3% w/v.

In some aspects the current disclosure also encompasses packaged sterile pharmaceutical composition for contacting an ocular surface of a mammal for the treatment of droopy eyes or red eyes, the sterile pharmaceutical composition comprising: a pharmaceutical agent, a cationic polymer, and a non-ionic polymer, wherein the application of the composition results in raising droopy eyelids and/or whitens the sclera for at least 6 hours.

In some aspects the packaged sterile pharmaceutical composition for contacting an ocular surface of a mammal for the treatment of droopy eyes or red eyes comprises at least two alpha-adrenergic agonists for example apraclonidine and oxymetazoline.

In some aspects the packaged sterile pharmaceutical composition for contacting an ocular surface of a mammal for the treatment of droopy eyes or red eyes does not comprise a preservative and is packaged in multi-dose bottles.

### DETAILED DESCRIPTION

It is to be understood that this disclosure is not limited to the particular methods, compositions, or materials specified herein, but is extended to equivalents thereof as would be recognized by those ordinarily skilled in the relevant arts. It should also be understood that terminology employed herein is used for the purpose of describing particular aspects only and is not intended to be limiting.

Concentrations, amounts, and other numerical data may be expressed or presented herein in a range format. It is to be understood that such a range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 2 to about 50” should be interpreted to include not only the explicitly recited values of 2 to 50, but also include all individual values and sub-ranges within the indicated range. Thus, included in this numerical range are individual values such as 2, 2.4, 3, 3.7, 4, 5.5, 10, 10.1, 14, 15, 15.98, 20, 20.13, 23, 25.06, 30, 35.1, 38.0, 40, 44, 44.6, 45, 48, and sub-ranges such as from 1-3, from 2-4, from 5-10, from 5-20, from 5-25, from 5-30, from 5-35, from 5-40, from 5-50, from 2-10, from 2-20, from 2-30, from 2-40, from 2-50, etc. This same principle applies to ranges reciting only one numerical value as a minimum or a maximum. Furthermore, such an interpretation should apply regardless of the breadth of the range or the characteristics being described.

As used herein, the term “about” is used to provide flexibility to a numerical range endpoint by providing that a given value may be “a little above” or “a little below” the endpoint. For example, the endpoint may be within 10%, 8%, 5%, 3%, 2%, or 1% of the listed value. Further, for the sake of convenience and brevity, a numerical range of “about 50 mg/mL to about 80 mg/mL” should also be understood to provide support for the range of “50 mg/mL to 80 mg/mL” The endpoint may also be based on the variability allowed by an appropriate regulatory body, such as the FDA, USP, etc.

As used herein, “comprises,” “comprising,” “containing,” and “having” and the like can have the meaning ascribed to them in U.S. Patent Law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. In this specification when using an open-ended term, like “comprising” or “including,” it is understood that direct support should be afforded also to “consisting essentially of” language as well as “consisting of” language as if stated explicitly and vice versa.

As used herein, “presbyopia” refers to a visual condition which typically first becomes apparent in a person's middle age in which loss of elasticity of the lens of the eye causes defective accommodation and an inability or a reduction in the ability to focus sharply for near vision.

As used herein, “myopia” is a type of refractive error, wherein light focuses in front of the retina rather than on the retina and includes but is not restricted to simple myopia, pre-myopia, early on-set myopia, high myopia, moderate myopia, low myopia, pseudomyopia, degenerative myopia, nocturnal myopia, induced myopia, index myopia, nearsightedness or short sightedness. Clinically for human's myopia refers to a patient's condition in which the patient has at least one eye with a spherical equivalent (SE) value greater than −0.5 D, for example, −1.0 D, −2.0 D. Depending on context, “myopia” also refers to the condition of the eye, the SE value of which is higher than −0.5 D. The term early onset myopia The term “pre-myopia” refers to a patient's condition in which the patient has at least one eye with an SE value within the range of −0.49 D to 1.00 D. Depending on context, myopic can also refer to the condition of the eye, the SE value of which is within the range of −0.49 D to 1.00 D. The term “low myopia” refers to a patient's condition in which the patient has at least one eye with an SE value within the range of −0.50 D to −1.50 D. Depending on context, “low myopia” can also refer to the condition of the eye, the SE value of which is within the range of −0.50 D to −1.50 D. The term “high myopia” refers to a person having at least one eye with an SE value that is greater than −5.0 D. Depending on context, “high myopia” can also refer to the condition of the eye, the SE value of which is greater than −5.0 D.

As used herein, the term “droopy eyes” or ptosis or blepharoptosis or lazy eye is the drooping or falling of the upper eyelid caused by the weakening of the levator and Muller muscles, or other conditions including but not limited to myogenic, neurogenic, neuromuscular (eg. Myasthenia gravis), aponeurotic, pseudoptosis, mechanical or traumatic causes. In may affect one or both eyes. It may be associated with other ocular conditions including but not restricted to hereditary, immunological, or degenerative disorders, tumors, stroke, age related weakness and infections. In may manifest itself in subjects of all ages.

As used here, the term “red eyes” is broadly used for an eye that appears red due to illness, condition or injury. It may result from multiple causes including for example eye irritation, blepharitis, medication, allergies, dry eye syndrome, conjunctivitis, subconjunctival hemorrhage, pterygium, pinguecula, episcleritis, scleritis, glaucoma, irititis, infections, trauma and/or injury.

As used herein, the term “subject” may include an animal, human or non-human, to whom treatment according to the methods of the present disclosure is provided. The compositions disclosed herein include compositions for treatment and/or prevention of ophthalmic conditions in subjects in need thereof. Human and veterinary applications are anticipated by the present disclosure. The term includes but is not limited to birds, reptiles, amphibians, and mammals, e.g., humans, other primates, pigs, rodents, such as mice and rats, rabbits, guinea pigs, hamsters, horses, cows, cats, dogs, sheep, chickens and goats. In some aspects, the subjects are humans, chickens, or mice. In some aspects, the subject is a human. Both pediatric and adult subjects are included. For example, in any of the methods described herein, the subject can be at least 6 months old (e.g., 6 months or older, 12 months or older, 18 months or older, 2 years or older, 4 years or older, 6 years or older, 10 years or older, 13 years or older, 16 years or older, 18 years or older, 21 years or older, 25 years or older, 30 years or older, 35 years or older, 40 years or older, 45 years or older, 50 years or older, 60 years or older, 65 years or older, 70 years or older, 75 years or older, 80 years or older, 85 years or older, 90 years or older, or 1, 2, 3, 4, 5, 6, 7, 8, 9, 10, 12, 13, 14, 15, 16, 18, 20, 21, 24, 25, 27, 28, 30, 33, 35, 37, 39, 40, 42, 44, 45, 48, 50, 52, 55, 60, 65, 70, 75, 80, 85, 90, 95, 96, 97, 98, 99, 100, 101, 102, 103, 104, or more years old).

In some aspects the term “subject” includes individuals that have been diagnosed with or are suspected of having or developing one or more ophthalmic disorders or conditions. These include but are not limited to refractive errors including myopia, hyperopia, astigmatism, presbyopia, age related macular degeneration, retinopathies, glaucoma, amblyopia, ptosis, red eyes, conjunctivitis, eye infections that include bacterial or viral infections, allergies or trauma.

In some aspects the term “subject” includes individuals that have been diagnosed with presbyopia. In some aspects subject may include individuals considered at risk for developing presbyopia who may benefit from the present disclosure, e.g., because prophylactic treatment can begin before there is any evidence and/or diagnosis of the disorder. Individuals “at risk” include, e.g., individuals having genetic or environmental factors associated with development of presbyopia, such as individuals having at least one hyperopic parent, individuals spending limited time outdoors, individuals with shorter axial lengths, and the like. Similarly, individuals in very early stages of presbyopia may benefit from prophylactic treatment.

In some aspects the term “subject” includes individuals that have been diagnosed with myopia. In some aspects subject may include individuals considered at risk for developing myopia or is pre-myopic who may benefit from the use of the methods and compositions of the present disclosures. Myopia may manifest itself in subjects of all ages. Subjects may suffer from myopia or be pre-myopic due to multiple factors including hereditary or environmental.

In some aspects the term “subject” includes individuals that have been diagnosed with droopy eyes, ptosis or blepharoptosis. In some aspects the subjects may suffer from droopy eyes due to underlying myogenic, neurogenic, aponeurotic, mechanical, age-related or traumatic causes. In some aspects the term subject includes individuals at risk of developing ptosis. In may affect one or both eyes of the subject. It may be associated with other ocular conditions in a subject including but not restricted to hereditary, immunological, or degenerative disorders, tumors, and infections. In may manifest itself in subjects of all ages.

Skilled practitioners will appreciate that a patient can be diagnosed, e.g., by a medical professional, e.g., a physician or nurse (or veterinarian, as appropriate for the patient being diagnosed), as suffering from or at risk for a condition described herein, e.g. refractive errors including myopia, hyperopia, astigmatism, presbyopia, age related macular degeneration, retinopathies, glaucoma, amblyopia, ptosis, red eyes, conjunctivitis, eye infections that include bacterial or viral infections, allergies or traumausing any method known in the art, e.g., by assessing a patient's medical history, performing diagnostic tests, and/or by employing imaging techniques. Various methods are known in the art to determine these exemplary conditions. Skilled practitioners will also appreciate that treatment need not be administered to a patient by the same individual who diagnosed the patient (or the same individual who prescribed the treatment for the patient). Treatment can be administered (and/or administration can be supervised), e.g., by the diagnosing and/or prescribing individual, and/or any other individual, including the patient her/himself (e.g., where the patient is capable of self-administration).

As used herein, “therapeutically effective quantity” refers to an amount that leads to measurable and beneficial effects for the subject administered the substance.

As used herein, the terms “treat,” “treating,” or “treatment” refer to both therapeutic treatment and prophylactic or preventative measures, wherein the object is to prevent or slow down (lessen) an undesired physiological change or disease/disorder. Beneficial or desired clinical results include, but are not limited to, alleviation of symptoms, diminishment of extent of disease, stabilized (i.e., not worsening) state of disease, a delay or slowing of disease progression, amelioration or palliation of the disease state, and remission (whether partial or total), whether detectable or undetectable. In the context of the present disclosure, beneficial or desired clinical results may include improvement in visual acuity as measured by a Jaeger eye test. “Treatment” can also mean prolonging survival as compared to expected survival if not receiving treatment. Those in need of treatment include those already with the disease, condition, or disorder as well as those prone to have the disease, condition or disorder or those in which the disease, condition, or disorder is to be prevented.

The current disclosure encompasses pharmaceutical compositions, deliverable packages with these compositions and methods of use of these compositions for treatment of eye conditions including but not limited to refractive errors including myopia, hyperopia, astigmatism, presbyopia, age related macular degeneration, retinopathies, glaucoma, amblyopia, ptosis, red eyes, conjunctivitis, eye infections that include bacterial or viral infections, allergies, or trauma. In some aspects the current disclosure encompasses sustained release formulations for ophthalmic drugs and pharmaceutical agents. Conventional methods of drug delivery to the eye (for instance topical drops) leads to spillage and are effective for a limited number (1-2 hrs) thus requiring multiple application for extended activity. Multiple applications have the multiple disadvantages including increasing patient noncompliance and greater exposure to undesirable components of the formulation.

The current disclosure results from the surprising discovery that formulating ophthalmic drugs with a combination of cationic polymers and non-ionic polymers can greatly increase retention and extend release times for the pharmaceutical agents. These polymers have properties specifically suited for ocular drug delivery. Such properties include controlled drug release, mucoadhesion, in situ gelation and enhanced corneal penetration as a results of longer residence time. They may also exhibit protective effects by reducing ocular surface damage. The compositions as provided herein have several benefits, key amongst which are sustained activity, high long-term stability, capability to include multiple pharmaceutical agents into one composition, lack of preservatives, low side effects, high long-term stability, low burning or stinging during ophthalmic use, low tear formation, and lack of hyperemia. These properties arise from the unique compositions described herein.

### I. Pharmaceutical Compositions

The pharmaceutical compositions of the current disclosure comprise at least a pharmaceutical agent, an ionic polymer, a non-ionic polymer and optionally excipients, wherein the composition provides sustained activity of the pharmaceutical agent over several hours post application. These compositions are essentially preservative free. These compositions are also envisaged to have low to negligible unreasonable side-effects.

### a) Pharmaceutical Agents

Described herein are ophthalmic compositions comprising a therapeutically effective quantity of a pharmaceutical agent. As used herein the term “pharmaceutical agent” also referred to as “drugs” or “therapeutic agents” refer to any chemical moiety that is a biologically, physiologically, or pharmacologically active substance that acts locally or systemically in a subject. Some examples of pharmaceutical agents are described in well-known literature references such as the Merck Index, the Merck manual of diagnosis and therapy, the Physicians' Desk Reference, and The Pharmacological Basis of Therapeutics, and they include, without limitation, proteins, peptides, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment. Various forms of a therapeutic agent may be used which are capable of being released from the subject composition into adjacent tissues or fluids upon administration to a subject.

In some exemplary aspects the compositions comprise one or more pharmaceuticals agents including but not restricted to cholinesterase inhibitors, mitotic agents including but not restricted to cholinesterase inhibitors, muscarinic receptor agonists, a (alpha)-1 adrenergic receptor antagonists, a (alpha)-2 adrenergic receptor agonists, B (beta)-adrenergic receptor antagonists, nicotine receptor agonists, adenosine receptor antagonists, antipsychotics, anti-emetics, cannabinoids, monoamine oxidase (MAO) inhibitors, Prostaglandin E2 receptor 1 (EP1) receptor agonists, Prostaglandin E2 receptor 4 (EP4) receptor agonists, and Prostaglandin F (FP) receptor agonists, pharmaceutical salts thereof, and combinations thereof.

### Cholinesterase Inhibitor

A composition of the present disclosure may comprise a cholinesterase inhibitor. Cholinesterase inhibitors, and methods for making or procuring cholinesterase inhibitors, are known in the art. Examples of cholinesterase inhibitors include but are not restricted to physostigmine, neostigmine, pyridostigmine, ambenonium, demecarium bromide, rivastigmine, galantamine, caffeine, rosmarinic acid, alpha-pinene, donepezil, tacrine, edrophonium, huperzine A, ladostigil, ungeremine, lactucopicrin, echothiophate, diisopropyl fluorophosphatesneostigmine, echothiophate iodide (also known as phospholine iodide), aceclidine, ambenonium, demecarium, rivastigmine, galantamine, acotiamide, diisopropyl fluorophsphate, cadusafos, chlorpyrifos, cyclosarin, dichlorvos, dimethoate, metrifonate, and any combination or pharmaceutically acceptable salts thereof. In some exemplary aspects, the cholinesterase inhibitor may be neostigmine or a pharmaceutically acceptable salt thereof. In some exemplary aspects, the cholinesterase inhibitor can include demecarium bromide or a pharmaceutically acceptable salt thereof. In some exemplary aspects, the cholinesterase inhibitor can include diisopropyl flurophosphate or a pharmaceutically acceptable salt thereof.

In some aspects, the concentration of the cholinesterase inhibitor in the composition may be between about 0.00001% (w/v) and about 2% (w/v). For instance, in some aspects the concentration of the at least one cholinesterase inhibitor may be about 0.00001% (w/v), 0.00005% (w/v), 0.0001% (w/v), 0.0005% (w/v), 0.001% (w/v), 0.005% (w/v), 0.01% (w/v), 0.05% (w/v), 0.1% (w/v), 0.5% (w/v), 1% (w/v), 1.5% (w/v), or 2% (w/v). Furthermore, in some aspects the concentration of the at least one cholinesterase inhibitor may be between about 0.00001% (w/v) to about 0.00005% (w/v), about 0.00005% (w/v) to about 0.0001% (w/v), about 0.0001% (w/v) to about 0.0005% (w/v), about 0.0005% (w/v) to about 0.001% (w/v), about 0.001% (w/v) to about 0.005% (w/v), about 0.005% (w/v) to about 0.01% (w/v), about 0.01% (w/v) to about 0.05% (w/v), about 0.05% (w/v) to about 0.1% (w/v), about 0.1% (w/v) to about 0.5% (w/v), about 0.5% (w/v) to about 1% (w/v), about 1% (w/v) to about 1.5% (w/v), or about 1.5% (w/v) to about 2% (w/v). In at least one example, the concentration of the at least one cholinesterase inhibitor is 0.1% (w/v).

### Muscarinic Receptor Agonist

A composition of the present disclosure may comprise a muscarinic receptor agonist. The muscarinic receptor agonist can be selected from the group consisting of pilocarpine, choline, acetylcholine, nicotine, methacholine, carbachol, cevimeline, CI-1017, bethanechol, milameline, muscarine, oxotremorine, sabcomeline, talsaclidine, tazomeline, vedaclidine, VU0152100, VU0238429, xenomeline, AF102B, AF150(S), AF267B, aceclidine, arecoline, pilocarpine, cevimeline, and any combination or pharmaceutically acceptable salts thereof.

In some aspects, the concentration of the muscarinic receptor agonist in the composition may be between about 0.001% (w/v) and about 1.0% (w/v). In some aspects the concentration of the muscarinic receptor agonist may be about 0.001%, 0.0025%, 0.005%, 0.01%, 0.025% (w/v), 0.05% (w/v), 0.075% (w/v), 0.1% (w/v), 0.25% (w/v), 0.5% (w/v), 0.75% (w/v), 1% (w/v), 1.25% (w/v), or 1.5% (w/v). Furthermore, in some aspects the concentration of the muscarinic receptor agonist may be between about 0.001% (w/v) to about 0.0025% (w/v), about 0.0025% (w/v) to about 0.005% (w/v), about 0.005% (w/v) to about 0.0075% (w/v), about 0.0075% (w/v) to about 0.01% (w/v), 0.01% (w/v) to about 0.025%, 0.025% (w/v) to about 0.05% (w/v), about 0.05% (w/v) to about 0.1% (w/v), about 0.1% (w/v) to about 0.25% (w/v), about 0.25% (w/v) to about 0.5% (w/v), about 0.5% (w/v) to about 0.75% (w/v), about 0.75% (w/v) to about 1% (w/v), about 1% (w/v) to about 1.25% (w/v), or about 1.25% (w/v) to about 1.5% (w/v).

### Muscarinic Receptor Antagonists

A composition of the present disclosure may comprise a muscarinic receptor antagonist. The muscarinic receptor antagonists can be selected from a group consisting of atropine, scopolamine, glycopyrrolate, ipratropium bromide and any other known muscarinic receptor antagonist used for treatment of myopia.

In some aspects, the concentration of the muscarinic receptor antagonist in the composition may be between about 0.025% (w/v) and about 3% (w/v). In some aspects the concentration of the muscarinic receptor agonist may be about 0.025% (w/v), 0.05% (w/v), 0.075% (w/v), 0.1% (w/v), 0.25% (w/v), 0.5% (w/v), 0.75% (w/v), 1% (w/v), 1.25% (w/v), 1.5% (w/v), 1.75% (w/v), 2% (w/v), 2.25% (w/v), 2.50% (w/v), 2.75% (w/v), or 3.0% (w/v). Furthermore, in some aspects the concentration of the muscarinic receptor agonist may be between about 0.025% (w/v) to about 0.05% (w/v), about 0.05% (w/v) to about 0.1% (w/v), about 0.1% (w/v) to about 0.25% (w/v), about 0.25% (w/v) to about 0.5% (w/v), about 0.5% (w/v) to about 0.75% (w/v), about 0.75% (w/v) to about 1% (w/v), about 1% (w/v) to about 1.25% (w/v), about 1.25% (w/v) to about 1.5% (w/v), about 1.5% (w/v) to about 1.75% (w/v), about 1.75% (w/v) to about 2% (w/v), about 2% (w/v) to about 2.25% (w/v), about 2.25% (w/v) to about 2.75% (w/v), or about 2.75% (w/v) to about 3.0% (w/v).

In some exemplary aspects, the compositions of the current disclosure may comprise the muscarinic receptor agonist atropine and pharmaceutically acceptable salts of atropine, used for treatment, prevention or delaying the onset of myopia (or other eye conditions) in a subject in need thereof.

### α-1-Adrenergic Receptor Antagonist

A composition of the present disclosure may comprise a α-1-adrenergic antagonist. α-1-adrenergic antagonists, and methods of making and procuring α-1-adrenergic antagonists, are known in the art. The α-1-adrenergic antagonist may include phenoxybenzamine, phentolamine, tolazoline, trazodone, alfuzosin, dapiprazole, thymoxamine, doxazosin, prazosin, tamsulosin, bunezosin, terazosin, trimazosin, silodosin, atipamezole, idazoxan, mirtazapine, yohimbine, carvedilol, labetalol, urapidil, abanoquil, adimolol, ajmalicine, amosulalol, arotinolol, atiprosin, benoxathian, buflomedil, bunazosin, carvedilol, CI-926, corynanthine, DL-017, domesticine, eugenodilol, fenspiride, GYM-12743, GYKI-16084, indoramin, ketanserin, L-765314, mephendioxan, metazosin, monatepil, naftopidil, nantenine, neldazosin, nicergoline, niguldipine, pelanserin, phendioxan, piperoxan, quinazosin, ritanserin, RS-97078, SGB-1534, SL-890591, spiperone, talipexole, tibalosin, tiodazosin, tipentosin, tolazoline, upidosin, zolertine, a pharmaceutically acceptable salt thereof, and combinations thereof, and any combination or pharmaceutically-acceptable salts thereof. In an exemplary aspect the at least one α-1-adrenergic antagonist includes phentolamine mesylate.

In some aspects, the concentration of the at least one α-1-adrenergic antagonist in the composition may be between about 0.001% (w/v) and about 1% (w/v). For instance, in some aspects the concentration of the at least one α-1-adrenergic antagonist in the composition may be about 0.001% (w/v), 0.002% (w/v), 0.003% (w/v), 0.004% (w/v), 0.005% (w/v), 0.006% (w/v), 0.007% (w/v), 0.008% (w/v), 0.009%, 0.01% (w/v), 0.02% (w/v), 0.03% (w/v), 0.04% (w/v), 0.05% (w/v), 0.06% (w/v), 0.07% (w/v), 0.08% (w/v), 0.09% (w/v), 0.1% (w/v), 0.2% (w/v), 0.3% (w/v), 0.4% (w/V), 0.5% (w/v), 0.6% (w/v), 0.7% (w/v), 0.8% (w/v), 0.9% (w/v), or about 1% (w/v). Furthermore, in some aspects the concentration of the at least one α-1-adrenergic antagonist in the composition may be between about 0.001% (w/v) to about 0.002% (w/v), about 0.002% (w/v) to about 0.003% (w/v), about 0.003% (w/v) to about 0.004% (w/v), about 0.004% (w/v) to about 0.005% (w/v), about 0.005% (w/v) to about 0.006% (w/v), about 0.006% (w/v) to about 0.007% (w/v), about 0.007% (w/v) to about 0.008% (w/v), about 0.008% (w/v) to about 0.009% (w/v), about 0.01% (w/v), 0.01% (w/v) to about 0.02% (w/v), about 0.02% (w/v) to about 0.03% (w/v), about 0.03% (w/v) to about 0.04% (w/v), about 0.04% (w/v) to about 0.05% (w/v), about 0.05% (w/v) to about 0.06% (w/v), about 0.06% (w/v) to about 0.07% (w/v), about 0.07% (w/v) to about 0.08% (w/v), about 0.08% (w/v) to about 0.09% (w/v), about 0.09% (w/v) to about 0.1% (w/v), about 0.1% (w/v) to about 0.2% (w/v), about 0.2% (w/v) to about 0.3% (w/v), about 0.3% (w/v) to about 0.4% (w/v), about 0.4% (w/v) to about 0.5% (w/v), about 0.5% (w/v) to about 0.6% (w/v), about 0.6% (w/v) to about 0.7% (w/v), about 0.7% (w/v) to about 0.8% (w/v), about 0.8% (w/v) to about 0.9% (w/v), or about 0.9% (w/v) to about 1% (w/v). In at least one example, the concentration of the at least one α-1-adrenergic antagonist is about 0.1% (w/v).

α-2-adrenergic receptor agonist

A composition of the present disclosure may include one or more a-2-adrenergic agonist. α-2-adrenergic agonists, as well as methods of making and procuring α-2-adrenergic agonists, are known in the art. The one or more α-2-adrenergic agonist may include apraclonidine, brimonidine, clonidine, mivazerol, naphazoline, oxymetazoline, tetrahydrozoline, guanfacine, guanabenz, guanoxabenz, guanethidine, xylazine, tizanidine, medetomidine, methyldopa, methylnorepinephrine, moxonidine, rilmenidine, fadolmidine, dexmedetomidine, amitraz, cannabivarin, detomidine, dihydroergotamine, dipivefrine, dopamine, ephedrine, ergotamine, esproquin, etilefrine, eEthylnorepinephrine, 6-fluoronorepinephrine, levonordefrin, lofexidine, naphazoline, 4-NEMD, (R)-3-nitrobiphenyline, norepinephrine, phenylpropanolamine, piperoxan, pseudoephedrine, rilmenidine, romifidine, talipexole, tetrahydrozoline, tolonidine, xylometazoline and combinations thereof, and any combination or pharmaceutically-acceptable salts thereof.

In some aspects, the concentration of the at least one α-2-adrenergic agonist in the composition may be between about 0.001% (w/v) and about 0.5% (w/v). For instance, in some aspects the concentration of the at least one α-2-adrenergic agonist in the composition may be about 0.001% (w/v), 0.002%, 0.003%, 0.004% 0.005% (w/v), 0.006%, 0.007%, 0.008%, 0.009% 0.01% (w/v), 0.02% (w/v), 0.03% (w/v), 0.04% (w/v), 0.05% (w/v), 0.06% (w/v), 0.07% (w/v), 0.08% (w/v), 0.09% (w/v), 0.1% (w/v), 0.2% (w/v), 0.3% (w/v), 0.4% (w/v), or about 0.5% (w/v). Furthermore, in some aspects the concentration of the at least one α-2-adrenergic agonist in the composition may be between about 0.001% (w/v) to about 0.005% (w/v), about 0.005% (w/v) to about 0.01% (w/v), about 0.01% (w/v) to about 0.02%, about 0.02% (w/v) to about 0.03% (w/v), about 0.03% (w/v) to about 0.04% about 0.04% (w/v) to about 0.05% (w/v), about 0.05% (w/v) to about 0.06% (w/v), about 0.06% (w/v) to about 0.07% (w/v), about 0.07% (w/v) to about 0.08% (w/v), about 0.08% (w/v) to about 0.09% (w/v), about 0.09% (w/v) to about 0.1% (w/v), about 0.1% (w/v) to about 0.2% (w/v), about 0.2% (w/v) to about 0.3% (w/v), about 0.3% (w/v) to about 0.4% (w/v), about 0.4% (w/v) to about 0.5% (w/v). In at least one example, the concentration of the at least one α-2-adrenergic agonist is about 0.1% (w/v).

### Cholinergic Agonist

A composition of the present disclosure may have at least one cholinergic agonist. Cholinergic agonists, as well as methods of making and procuring cholinergic agonists, are known in the art. The at least one cholinergic agonist may include arecoline, muscarine, pilocarpine, bethanechol, carbachol, cevimeline, methacholine, and any combination or pharmaceutically acceptable salts thereof.

In some aspects, the concentration of the at least one cholinergic agonist in the composition may be between about 0.1% (w/v) and about 4% (w/v). For instance, in some aspects the concentration of the at least one cholinergic agonist in the composition is about 0.1% (w/v), 0.25% (w/v), 0.5% (w/v), 0.75% (w/v), 1% (w/v), 1.25% (w/v), 1.5% (w/v), 1.75% (w/v), 2% (w/v), 2.25% (w/v), 2.5% (w/v), 2.75% (w/v), 3% (w/v), 3.25% (w/v), 3.5% (w/v), 3.75% (w/v), or about 4% (w/v). Furthermore, the concentration of the at least one cholinergic agonist in the composition may be between about 0.1% (w/v) to about 0.25% (w/v), about 0.25% (w/v) to about 0.5% (w/v), about 0.5% (w/v) to about 0.75% (w/v), about 0.75% (w/v) to about 1% (w/v), about 1% (w/v) to about 1.25% (w/v), about 1.25% (w/v) to about 1.5% (w/v), about 1.5% (w/v) to about 1.75% (w/v), about 1.75% (w/v) to about 2% (w/v), about 2% (w/v) to about 2.25% (w/v), about 2.25% (w/v) to about 2.5% (w/v), about 2.5% (w/v) to about 2.75% (w/v), about 2.75% (w/v) to about 3% (w/v), about 3% (w/v) to about 3.25% (w/v), about 3.25% (w/v) to about 3.5% (w/v), about 3.5% (w/v) to about 3.75% (w/v), or about 3.75% (w/v) to about 4% (w/v). In at least one example, the concentration of the at least one cholinergic agonist is about 0.55%.

### Other Pharmaceutical Agents

In addition to the pharmaceutical agents described thus far, compositions in the current disclosure may comprise any ophthalmic pharmaceutical agent known in the art that can be compounded with a cationic polymer, a non-ionic polymer and pharmaceutical carriers to provide beneficial outcomes. Non-limiting examples of these pharmaceutical agents include β(beta)-adrenergic receptor antagonists, nicotine receptor agonists, adenosine receptor antagonists, antipsychotics, anti-emetics, cannabinoids, monoamine oxidase (MAO) inhibitors, Prostaglandin E2 receptor 1 (EP1) receptor agonists, Prostaglandin E2 receptor 4 (EP4) receptor agonists, and Prostaglandin F (FP) receptor agonists, antibiotics, pharmaceutical salts thereof, and combinations thereof.

Non-limiting examples of β-adrenergic receptor antagonists include acebutolol, atenolol, betaxolol, bisoprolol, carteolol esmolol, isoproterenol, levobunolol, metoprolol, penbutolol nadolol, nebivolol, pindolol, propranolol, timolol, sotalol, and the like, and/or a pharmaceutically acceptable salt of any one or more of the preceding compounds.

Non-limiting examples of nicotine receptor agonist include nicotine, varenicline, galantamine, epibatidine, lobeline, decamethonium, cytosine, nifene, dimethylphenylpiperazinium, and the like, and/or a pharmaceutically acceptable salt of any of the preceding compounds, or a combination thereof.

Non-limiting examples of adenosine receptor antagonists include 7-methylxathine, caffeine, theophylline, theobromine, and the like, and/or a pharmaceutically acceptable salt of any of the preceding compounds, or a combination thereof.

Non-limiting examples of antipsychotics include risperdal, haloperidol, thorazine, olanzapine, quetiapine, mirtazapine, chlorpromazine, prochlorperazine, alizapride, metoclopramide, midazolam, lorazepam, and the like, and/or a pharmaceutically acceptable salt of any of the preceding compounds, or a combination thereof.

Non-limiting examples of anti-emetics include ondansetron, droperidol, metoclopramide, dolasetron, granisetron, tropisetron, palonosetron, domperidone, aprepitant, casopitant, rolapitant, cyclizene, diphenhydramine, dimenhydinate, doxylamine, meclizine, promethazine, hydroxylzine, and the like, and/or a pharmaceutically acceptable salt of any one or more of the preceding compounds.

Non-limiting examples of cannabinoids include cannabis, dronebinol, nabilone, sativex, and the like, and/or a pharmaceutically acceptable salt of any of the preceding compounds, or a combination thereof.

Non-limiting examples of monoamine oxidase (MAO) inhibitors include selegiline, befloxatone moclobemide, isocarboxazid, nialamide, pheneizine, hydracarbazine, traylcypromine, bifemelane, pirlindole, toloxatone, rasagiline, linezolid, methylene blue, and the like, and/or a pharmaceutically acceptable salt of any of the preceding compounds, or a combination thereof.

Non-limiting examples of EP1 receptor agonists, EP4 receptor agonists, and FP receptor agonists include PGE2, PGE1, PGF2a, PGD2, PGE2, PGI2, TXA2, cloprostenol, flupostenol, latanoprost, tafluprost, enprostil, sulprostone, U46619, carbacyclin, and iloprost, ONO-D1-004, 1-hydroxy-PGE1, rivenprost (ONO-4819), OOG-308, ONO-AE1-329, AGN205203, ONO-4819, CP-734432, AE1-329, SC-19220, SC-51089, EP4RAG, and the like, and/or a pharmaceutically acceptable salt of any of the preceding compounds, or a combination thereof.

Non-limiting examples of other miotic agents useful in the compositions described herein include camptothecin (which is generally known as a cancer drug) and derivatives, ionomysin (Ca2+ channel modulator), thapsigargin (Ca2+ channel modulator), reserpine (norepinephrine depleting agents), and the like, and/or a pharmaceutically acceptable salt of any of the preceding compounds, or a combination thereof. Analogs of the miotic agents that function as miotic agents are also embraced by the compositions and methods described herein. The ability of such analogs to exhibit miotic effects can be tested using methods known in the art.

Non-limiting examples of antibiotics useful in these compositions include Neomycin, polymyxin, bacitracin and/or a pharmaceutically acceptable salt of any of the preceding compounds, or a combination thereof.

In some aspects, the concentration of these pharmaceutical agents in the compositions may range from about 0.001% (w/v) to about 10% (w/v).

In some aspects, the current disclosure encompasses compositions with a single pharmaceutical agent. In some aspects the composition may comprise more than one pharmaceutical agent or drug. In some aspects the current disclosure encompasses compositions comprising one, two, three, or more drugs compounded in a single composition with a cationic polymer, a non-ionic polymer and excipients.

The pharmaceutical agents described herein may be administered per se or in the form of a pharmaceutically acceptable salt. When used in a formulation, the salts should be both pharmacologically and pharmaceutically acceptable, but non-pharmaceutically acceptable salts may be conveniently used to prepare the active free compound or pharmaceutically acceptable salts thereof. A pharmaceutically acceptable salt can be any salt that retains the activity of the parent compound and does not impart any additional deleterious or untoward effects on the subject to which it is administered and in the context in which it is administered compared to the parent compound. Pharmaceutically acceptable salts of acidic functional groups may be derived from organic or inorganic bases. The salt may comprise a mono or polyvalent ion. Of particular interest are the inorganic ions, lithium, sodium, potassium, calcium, and magnesium. Organic salts may be made with amines, particularly ammonium salts such as mono-, di- and trialkyl amines or ethanol amines. Salts may also be formed with caffeine, tromethamine and similar molecules. Hydrochloric acid or some other pharmaceutically acceptable acid may form a salt with a compound that includes a basic group, such as an amine or a pyridine ring. In some embodiments, pharmaceutically acceptable salts can also include salts of active compounds which are prepared with relatively nontoxic acids or bases, depending on the particular substituent moieties found on the compounds described herein. When compounds useful in the compositions and methods described herein contain relatively acidic functionalities, base addition salts can be obtained by contacting the neutral form of such compounds with enough of the desired base, either neat or in a suitable inert solvent. Examples of pharmaceutically acceptable base addition salts include sodium, potassium, calcium, ammonium, organic amino, or magnesium salt, or a similar salt. When compounds useful in the compositions and methods described herein contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66:1-19) which is incorporated by reference. In some aspects, compounds useful in the compositions and methods described herein may contain both basic and acidic functionalities that allow the compounds to be converted into either base or acid addition salts.

### b) Cationic Polymer

In some aspects the current disclosure encompasses pharmaceutical compositions comprising pharmaceutical agents described herein compounded with cationic polymers. Addition of cationic polymers provides surprising and remarkable benefits to the pharmaceutical compositions provided herein. These include but are not restricted to increasing wettability, enhancing stability, uniform and controlled release rates, decreasing the adherence of unwanted antigens, cells, proteins, reducing burning, stinging and tearing. Additionally, the positive charge on the polymer interacts with negatively charged glycosaminoglycan layer in the eye to create mucoadhesion. “Cationic polymers” as used herein are macromolecules that bear positive charges, which can be either intrinsically present in the polymer backbone and/or in the side chains. Most cationic polymers possess primary, secondary or tertiary amine functional groups that can be protonated. They also differ widely in their polymeric structure (linear, branched, hyperbranched and dendrimer-like) and can be further differentiated by the placement of the positive charges (backbone or side chains). The cationic polymers may be natural, semi-synthetic and synthetic in origin. Examples of cationic polymers include but are not restricted to chitosan, a chitosan derivative, a cationic (meth) acrylate copolymer, a cationic silicone polymer, a diallyl quaternary ammonium salt-acrylamide copolymer, cationic hydrolyzed keratin, cationic hydrolyzed silk, cationic hydrolyzed collagen, cationic hydrolyzed casein, cationic hydrolyzed soy protein, a cationic vinylpyrrolidone copolymer, poly l-lysine, dimethyldiacrylammonium chloride homopolymer, an adipic acid-dimethylaminohydroxypropyldiethylenetriamine copolymer, an adipic acid-epoxypropyldiethylenetriamine copolymer and an acrylamide-β-methacryloyloxyethyltrimethylammoniummethyl sulfate copolymer.

In some aspects, the concentration of the cationic polymer may be between 0.001 to 10% (w/v). For instance, in some aspects the concentration of the cationic polymer may be about 0.001% (w/v), 0.005% (w/v), 0.01% (w/v), 0.05% (w/v), 0.1% (w/v), 0.5% (w/v), 1% (w/v), 1.5% (w/v), 2% (w/v), 3% (w/v), 4% (w/v), 5% (w/v), 6% (w/v), 7% (w/v), 8% (w/v), 9% (w/v), or 10% (w/v). Furthermore, in some aspects the concentration of the cationic polymer may be between about 0.001% (w/v) to about 0.005% (w/v), about 0.005% (w/v) to about 0.01% (w/v), about 0.01% (w/v) to about 0.05% (w/v), about 0.05% (w/v) to about 0.1% (w/v), about 0.1% (w/v) to about 0.5% (w/v), about 0.5% (w/v) to about 1% (w/v), about 1% (w/v) to about 1.5% (w/v), or about 1.5% (w/v) to about 2% (w/v). In some exemplary aspects the cationic polymer has a concentration of about 0.01% w/v to about 2.0% w/v.

### c) Non-Ionic Polymer

In some aspects the current disclosure encompasses pharmaceutical compositions comprising pharmaceutical agents described herein and non-ionic polymers. Like cationic polymers, the use of non-ionic polymers in these formulations were found to provide several benefits. This includes improved drug retention within the eye, uniform and controlled release rates, reduced burning, stinging, tearing, hyperemia and greater stability during storage, and flexibility in dosing and drug delivery. Non-ionic polymers also form a protective layer on the ocular surface. The term “non-ionic polymer” as used herein refers to macromolecules with repeating units with no net charge. In some aspects the polymer is a natural non-ionic polymer. In some aspects the polymer is a synthetic non-ionic polymer. In certain aspects, the polymer is a non-ionic block copolymer. In some embodiments, the polymer may be a copolymer, e.g., where one repeat unit is relatively hydrophobic, and another repeat unit is relatively hydrophilic. The copolymer may be, for example, a diblock, triblock, alternating, or random copolymer. Non-limiting examples of non-ionic polymers include cellulose and cellulose derivatives, polysaccharides, guar-gum, pullulan, hydroxypropylcellulose, carboxymethylcellulose (CMC), polyvinyl pyrrolidone (eg. Povidone K30), poly (oxyethylene-co-oxypropylene) (eg. Poloxamer 407), polyacrylic acid, polyethylene glycols, polyester, polycaprolactone, poly (D,L-lactic-co-glycolic acid) (PLGA), poly lactic acid (PLA), polyurethane, poly glycolic acid (PGA) dextrans, Tween 20, Tween 80, Cremophor EL, and poloxamer.

In some aspects, the concentration of the non-ionic polymer may be between 0.001 to 10% (w/v). For instance, in some aspects the concentration of the non-ionic polymer may be about 0.001% (w/v), 0.005% (w/v), 0.01% (w/v), 0.05% (w/v), 0.1% (w/v), 0.5% (w/v), 1% (w/v), 1.5% (w/v), 2% (w/v), 3% (w/v), 4% (w/v), 5% (w/v), 6% (w/v), 7% (w/v), 8% (w/v), 9% (w/v), or 10% (w/v). Furthermore, in some aspects the concentration of the non-ionic polymer may be between about 0.001% (w/v) to about 0.005% (w/v), about 0.005% (w/v) to about 0.01% (w/v), about 0.01% (w/v) to about 0.05% (w/v), about 0.05% (w/v) to about 0.1% (w/v), about 0.1% (w/v) to about 0.5% (w/v), about 0.5% (w/v) to about 1% (w/v), about 1% (w/v) to about 1.5% (w/v), or about 1.5% (w/v) to about 2% (w/v). In some exemplary aspects the non-ionic polymer has a concentration of about 0.01% w/v to about 2.0% w/v.

### e) Excipients

In some aspects the pharmaceutical compositions provided herein may further comprise pharmaceutically-acceptable excipients, including salts, solvents, pH adjusting agents, buffering agents, antioxidants, tonicity modifying agents (including chloride salts), osmotic adjusting agents, preservatives, antibacterial agents, stabilizing agents, viscosity adjusting agents, surfactants, or any pharmaceutically-acceptable excipient known in the art or combinations thereof.

Exemplary non-limiting salts which can be used include sodium chloride, zinc sulfate, and potassium chloride. A salt can be used in any appropriate concentration. For example, salt (e.g., sodium chloride) can be used in a concentration of about 0.1% to about 0.5% (w/v) (e.g., about 0.2% to about 0.4%, about 0.1% to about 0.3%, about 0.3% to about 0.5%, about 0.1%, about 0.2%, about 0.3%, about 0.4%, or about 0.5% (w/v)).

Exemplary non-limiting buffers which can be used include boric acid and citric acid. A buffer can be used in any appropriate concentration. For example, a buffer (e.g., boric acid) can be used in a concentration of about 0.5% to about 2.0% (w/v) (e.g., about 0.5% to about 1.0%, about 1.0% to about 1.5%, about 1.5% to about 2.0%, about 1% to about 2%, about 0.5% to about 1.5%, about 0.75% to about 1.25%, about 0.9% to about 1.1%, about 0.5%, about 0.75%, about 1.0%, about 1.25%, about 1.5%, about 1.75%, or about 2.0% (w/v)).

In some aspects the formulations of the current disclosure are essentially preservative free. The formulations lack preservatives like benzalkonium chloride, phenolic compounds, quaternary ammonium compounds, and other commonly used preservatives used in ocular formulations.

Exemplary non-limiting viscosity modifiers which can be used include methyl cellulose, carboxymethyl cellulose, hydroxypropylmethyl cellulose, glycerol, and polyethylene glycol. A viscosity modifier can be used in any appropriate concentration.

Exemplary non-limiting stabilizing agents include sodium thiosulfate, sodium metabisulfite and disodium EDTA. In some aspects disodium EDTA may be added at a concentration of about 0.05 mg/mL to about 1 mg/mL. As used in the formulations described herein EDTA is not used as a preservative but as a stabilizing agent.

Exemplary non-limiting tonicity agents include glycerin, chloride salts of: sodium, potassium, magnesium, or calcium.

Exemplary non-limiting osmotic agents which can be used include mannitol and sorbitol. An osmotic agent can be used in any appropriate concentration Exemplary non-limiting delivery enhancing agents that facilitate the delivery of the therapeutic compound(s) of the disclosure into the aqueous humor, include substances which increase corneal permeability, such as surfactants (e.g., an anionic surfactant, a nonionic surfactant, or a combination thereof), solubilizers, wetting agents, liposomes, DMSO, and the like, each of which can be used in any appropriate concentration. Exemplary solubilizers include cyclodextrins (e.g., a gamma cyclodextrin, a beta-cyclodextrin (e.g., sulfobutylether β-cyclodextrin or 2-hydroxypropyl beta-cyclodextrin)). Exemplary anionic surfactants include sodium lauryl sulfate and sodium ester lauryl sulfate. Exemplary examples of anionic polymers include polycarbophil and carbomer. Exemplary nonionic surfactants include a poloxamer, a polysorbate, sorbitan monolaurate, sorbitan monopalmitate, sorbitan monostearate, sorbitan monooleate, polyoxyl stearate (e.g., polyolyl 40 stearate), a polyoxyl alkyl, and derivatives thereof.

### f) Exemplary Formulations

### Formulation I

i. A pharmaceutically-acceptable formulation was prepared as described below. The following products were used in the amounts and concentrations specified:
ii. about 0.010 g of atropine sulfate monohydrate;
iii. about 0.20 g of poloxamer 407;
iv. about 1.00 g of polyvinylpyrrolidone (povidone k30);
v. about 0.10 g of EDTA disodium;
vi. about 0.500 g of sodium chloride;
vii. about 0.500 g of glycerin;
viii. about 100 ml of water for injection.

### Formulation II

i. A pharmaceutically-acceptable formulation was prepared as described below. The following products were used in the amounts and concentrations specified:
ii. about 0.010 g of atropine sulfate monohydrate;
iii. about 0.10 g of chitosan hydrochloride;
iv. about 1.00 g of polyvinylpyrrolidone (povidone k30);
V. about 0.10 g of EDTA disodium;
vi. about 0.500 g of sodium chloride;
vii. about 0.500 g of glycerin;
viii. about 100 ml of water for injection.

### II. Formulation Properties and Use

In some aspects the current disclosure encompasses pharmaceutical formulations for ophthalmic use. In some aspects these compositions may be used for treatment of a broad range of ophthalmic disorders including but not limited refractive errors including myopia, hyperopia, astigmatism, presbyopia, age related macular degeneration, retinopathies, glaucoma, amblyopia, ptosis, red eyes, conjunctivitis, eye infections that include bacterial or viral infections, allergies or trauma. In some exemplary aspects these formulations are useful in the treatment of presbyopia, myopia, droopy or red eye. In some aspects, the current disclosure encompasses methods of using the formulations provided herein to provide sustained release of the formulated pharmaceutical agent over several hours. In some aspects the current formulations also provide additional benefits in comparison to conventionally formulated treatments for ophthalmic conditions like low burning, low stinging sensation, low tear formation, less redness, reduced pupil constriction, reduced light sensitivity and lack of hyperemia. These formulations have high long term stability during storage and use and are essentially preservative free.

### Physical Characteristics

In some aspects the compositions disclosed here may be formulated into topical gels, topical liquids, liquid eye drops, suspensions, creams, ointments, sprays, powders, solid implants for ophthalmic use.

In some aspects the formulation can be applied in a liquid carrier. In some aspects the carrier is an aqueous carrier. In some aspects the compositions may be formulated with carriers that impart high viscosity to the formulation to be used as gels or ointments. Examples of gelling carriers include but are not restricted to guar gum, locust bean gum, carob gum, Tara gum, crosslinking agents, gellan, thermogels, carrageenan, carbomers, galactomannan. Based on the carriers the viscosity of the formulations can vary from about 10 cps to about 200,000 cps. In some aspects the viscosity of the formulation can be about 10 cps to about 1000 cps, or about 1000 cps to about 10,000 cps, or about 10,000 cps to about 100,000 cps, or about 100,000 cps to about 200,000 cps.

In some aspects, the ophthalmic pharmaceutical formulation may have a pH of about 5.0 to 7.5. In some aspects, the pH may be about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5. In some additional aspects, the pH may be from about 5.0 to about 5.5 or about 5.5 to about 6.0. In some examples, the pH may be from about 5.0-5.5, about 5.5-6.0, about 6.0-6.5, about 6.5-7.0, or about 7.0-7.5. In some additional examples, the pH may be about 5.2.

In some aspects the sterile pharmaceutical compositions provided herein have high stability during storage. The high stability allows storage of sterile packaged products with current formulations for at least over 180 days at controlled room temperature of 25° C. (±2) and 60% (±5%) relative humidity. As used herein the term “stable” means that the sterile packaged formulation is potent and active as determined by a suitable assay on storage. More specifically the active concentration of the pharmaceutical agent is within ±10% of the original packaged formulation. In some aspects the concentration of the pharmaceutically active agent is within about ±1%, about ±2%, about ±3%, about ±4%, about ±5%, about ±6%, about ±7%, about ±8%, about ±9%, or about ±10% of the original packaged formulation during storage for about 6 months. In some aspects the sterile composition has a stable pH for at least 6 months during storage, more specifically the pH remains within ±0.5 of the pH of the originally packaged formulation. In some aspects the sterile composition has low acceptable levels of impurities during the duration of storage for about 6 months. In some aspects the formulation is stable and within acceptable criteria as set by the FDA or other suitable agencies for about 6 months, or about 7 months, or about 8 months, or about 9 months, or about 10 months, or about 11 months, or about 12 months, or more. In some exemplary aspects where the pharmaceutical agent comprises atropine sulfate, composition of the formulations comprises less than or equal to 0.002 mg/ml of tropic acid for the duration of 6 months of storage at controlled room temperature of 25° C. (±2) and 60% (±5%) relative humidity and the active concentration is within 10% of the original concentration of atropine sulfate and the pH remains in the range of 5.7+0.5 during the duration of the storage.

### Therapeutic Characteristics and Method of Use

Also provided herein are methods comprising administering to an eye of a subject, during a treatment period, the pharmaceutical formulations provided herein. In some aspects, provided herein are methods comprising administering to an eye of a subject, during a treatment period, the disclosed compositions. In some embodiments, the methods are methods of treating for example presbyopia, myopia, droopy or red eyes in a subject and comprise administering to an affected eye of the subject a therapeutically effective amount of the disclosed formulations while minimizing one or more side effects such as, e.g., pupil constriction, light sensitivity, hyperemia, redness, swelling, itchiness, burning and any combination thereof in the subject.

In some aspects the current formulations reduce droopy eyes or red eyes. In some aspects the current formulations result in raising droopy eyelids and/or whitening of sclera.

Exemplary methods include administering to a subject in need of treatment therapeutically effective amounts of any of the compositions or formulations described herein comprising a cholinesterase antagonist, a miotic agent, or a combination thereof compounded with cationic and non-ionic polymers. Some exemplary methods include administering to the subject a therapeutically effective amount of a cholinesterase antagonist compounded with cationic and non-ionic polymers. Some exemplary methods include administering to the subject a therapeutically effective amount of a miotic agent compounded with cationic and non-ionic polymers. Other exemplary methods include administering to the subject therapeutically effective amounts of each of a cholinesterase antagonist and a miotic agent compounded with cationic and non-ionic polymers. In some aspects of the methods described herein, the cholinesterase antagonist and the miotic agent thereof compounded with cationic and non-ionic polymers can be administered concurrently. In some aspects of the methods described herein, the cholinesterase antagonist and the miotic agent thereof compounded with cationic and non-ionic polymers can be administered subsequently. In some aspects of the methods described herein, the cholinesterase antagonist compounded with cationic and non-ionic polymers can be administered prior to administering the miotic agent compounded with cationic and non-ionic polymers. In some aspects of the methods described herein, the cholinesterase antagonist can be administered after the miotic agent compounded with cationic and non-ionic polymers. In various aspects of the methods described herein, the methods can treat disease in the subject. In some aspects of the methods described herein, the cholinesterase antagonist, the miotic agent, or the combination thereof is administered to an affected eye of the subject.

In some aspects of any of the methods described herein, the subject has or is at risk for developing presbyopia in at least one eye. For example, in some aspects, methods described herein can also be useful for treatment of presbyopia. As another example, in some aspects, methods described herein can also be useful for prophylactic treatment of presbyopia. As used herein, treating includes “prophylactic treatment”, which means reducing the incidence of or preventing (or reducing the risk of) a sign or symptom of a disease (e.g., presbyopia) in a subject at risk of developing a disease (e.g., presbyopia). The method described herein is suited particularly for subjects who are otherwise free of indications for ophthalmic treatments utilizing a cholinesterase antagonist and/or a miotic agent. In some aspects of the methods described herein, the cholinesterase antagonist, miotic agent, or combination thereof is administered in an amount sufficient to inhibit, slow, or prevent progression of presbyopia in the eye. In some aspects of the methods described herein, the cholinesterase antagonist, miotic agent, or combination thereof is administered in an amount sufficient to inhibit or slow shrinkage in the axial length of the eye.

In some aspects of the methods described herein, the subject has or is at risk for developing myopia in at least one eye. For example, in some aspects, methods described herein can also be useful for treatment of myopia. As used herein, treating includes “prophylactic treatment”, which means reducing the incidence of or preventing (or reducing the risk of) a sign or symptom of a disease (e.g., myopia) in a subject at risk of developing a disease (e.g., myopia). In some aspects, the methods described herein include administration of compositions comprising a muscarinic receptor antagonist like atropine or pharmaceutically acceptable salts thereof to treat, slow or prevent myopia. In some aspects, administering the composition to a subject in need thereof, e.g., a subject having pre-myopia, reduces the change in refraction by at least 10%, at least 20%, at least 30%, or at least 40%, or at least 50%, e.g., at least 53% over a period of 2 weeks, 1 month, 2 months, 6 months, one year, two years or more from the initiation of the treatment. In some embodiments, treating the subject, e.g., a subject having pre-myopia, reduces the increase of Axial length by at least 10%, at least 15%, at least 20%, at least 30% over a period of 1 week, 2 weeks, 1 month, 2 months, 6 months, one year, two years or more from or more, from the initiation of the treatment. In some embodiments, treating the patient, e.g., a patient having pre-myopia, with the composition disclosed herein, can reduce change in refraction by at least 10%, at least 20%, at least 30%, or at least 40%, or at least 50%, or at least 60%, or at least 70%, or at least 80%. In some aspects, administration of the atropine/muscarinic receptor antagonists or pharmaceutically acceptable salts thereof reduces the change in refraction (i.e., myopic refractive error shift) by at least 10%, e.g., at least 20%, at least 30%, or at least 40%, at least 50%, or at least 60%, or at least 70%, or at least 80% as compared to controls. In some embodiments, treating the patient, e.g., a patient having pre-myopia, with the composition disclosed herein reduces the rate of myopia progression or myopia shift by at least 10%, e.g., at least 20%, at least 30%, or at least 40%, at least 50%, or at least 60%, or at least 70%, or at least 80% after onset of myopia as compared to controls. In some embodiments, treating the patient with the composition disclosed herein increases the length of the time period from initiation of the treatment to onset of myopia by at least 10%, e.g., at least 20%, at least 30%, or at least 40%, at least 50%, or at least 60%, or at least 70%, or at least 80%, as compared to controls.

In some aspects the methods described herein, the subject has or is at risk of having or worsening droopy eyes or red eyes. In some aspects the compositions provided here may comprise a-adrenergic agonists that may be used for long or short term treatment of droopy eyes or red eyes.

In some aspects, methods described herein can include treating the eye with an ocular device during the treatment period. In some aspects, the ocular device can be a lens, such as a contact lens, an implanted lens, or a lens associated with external devices such as glasses. In some aspects the ocular device can be a corrective lens. In some aspects, a contact lenses or implantable lenses may contain or be treated with any of the compositions described herein to provide a route of administration for the compositions.

In some aspects, the methods described herein can result in a decreased need (e.g., in terms of frequency of use, prescription strength, or need for bifocals) or removal of need for an ocular device (e.g., glasses or contact lenses) during the treatment period. For example, a subject treated with a composition or formulation described herein may have a decreased need of using an ocular device (e.g., as compared to such a need before treatment) during the treatment period. As another example, a subject treated with a composition or formulation described herein may not need an ocular device (e.g., as compared to such a need before treatment) during the treatment period.

In some aspects the therapeutic compositions disclosed herein facilitate sustained or extended release of the pharmaceutical agent. As used herein the terms “sustained release” or “extended release” or “long-acting” allows delivery of the pharmaceutical agent at a desirable rate that leads to drug delivery for a prolonged period of time. In some aspects of the current disclosure, the pharmaceutical agent is active after application to the eye of the subject for greater than 6 hours. In some aspects the pharmaceutical agent is active after application to the eye of the subject for greater than 7, or 8, or 9, or 10, or 11, or 12, or 13, or 14, or 15, or 16, or 17, or 18, or 19, or 20, or 21, or 22, or 23, or 24 hours, or 1 day, or 2, or more days. In some aspects the current formulations are active on application for longer than conventional formulations with equal dosage of the drug. In some aspects the current formulations reduce droopy eyes or red eyes. In some aspects the current formulations result in raising droopy eyelids and/or whitening of sclera for at least 6 hours. In some aspects the disclosed formulations can reduce droopy eyes and redness for about 6, or about 7, or about 8, or about 9, or about 10, or about 11, or about 12, or about 13, or about 14, or about 15, or about 16, or about 17, or about 18, or about 19, or about 20, or about 21, or about 22, or about 23, or about 24 hours, or about 1 day, or about 2, or more days.

In some aspects the current disclosure encompasses formulations that may cause reduced hyperemia in comparison to some or all currently available formulations with equivalent drug dosage. In some aspects the use of a combination of cationic polymer and non-ionic polymer in these formulations and the lack of certain excipients and preservatives like benzalkonium chloride, phenolic compounds and quaternary ammonium compounds reduces the burning and stinging caused by application of the disclosed formulation. In some additional aspects the use of the current formulation also exhibits reduced redness of the eye, reduced blood flow, reduced itching, inflammation and other undesirable side effects compared to some or all of the conventionally formulated with equivalent drug dosage.

Dosage levels will vary greatly depending upon the individual to be treated, the progression of the disorder, the and the specific pharmaceutical agent(s) used. One of ordinary skill in the art, such as a health care provider, can determine proper dosing without undue experimentation and according to standard procedures in the art. Exemplary dosage amounts useful in some embodiments of the methods described herein include 1-2 drops per application. In some embodiments, drop sizes range from about 30 μL to about 80 μL. In some embodiments, exemplary dosage amounts can range from about 30 μL to about 480 μL per application. In some aspects these formulations are designed to be effective for at least 6 hrs after application. As such, in some aspects of the current disclosure the dosage is consolidated into 3 dosage per day, 2 dosage per day, one dosage per day, one dosage every other day, one dosage per week, two dosage per week, or three dosage per week.

In some embodiments, compositions or formulations described herein can be administered in an application having an amount of from about 1 μL to about 480 μL per application. In some embodiments, application amounts can range from about 10 μL to about 400 μL, from about 20 μL to about 300 μL, from about 20 μL to about 250 μL, from about 20 μL to about 200 μL, from about 20 μL to about 150 μL, from about 20 μL to about 100 μL, from about 25 μL to about 90 μL, from about 25 μL to about 85 μL, from about 30 μL to about 80 μL, from about 25 μL to about 50 μL, from about 25 μL to about 45 μL, from about 25 μL to about 40 μL, from about 30 μL to about 40 μL, from about 55 μL to about 90 μL, from about 60 μL to about 85 μL, or from about 60 μL to about 80 μL per application.

In some embodiments, compositions or formulations described herein (e.g., compositions or formulations including the cholinesterase antagonist, the miotic agent, or a combination thereof) can be administered during a treatment period. In some aspects, exemplary treatment periods include 1 day, up to about 5 days, up to about 10 days, up to about 30 days, up to about 1 week, up to about 2 weeks, up to about 3 weeks, up to about 4 weeks, up to about 5 weeks, up to about 1 month, up to about 2 months, up to about 3 months, up to about 4 months, up to about 5 months, up to about 6 months, up to about 7 months, up to about 8 months, up to about 9 months, up to about 10 months, up to about 11 months, up to about 1 year, up to about 2 years, up to about 3 years, up to about 4 years, up to about 5 years, or up to about 10 years, from about 1 day to about 10 years, from about 1 month to about 10 years, from about 2 months to about 10 years, from about 3 months to about 10 years, from about 4 months to about 10 years, from about 5 months to about 10 years, from about 6 months to about 10 years, from about 6 months to about 9 years, from about 6 months to about 8 years, from about 6 months to about 7 years, from about 6 months to about 6 years, from about 6 months to about 5 years, from about 1 day to about 60 months, from about 6 months to about 4 years, from about 6 months to about 3 years, from about 6 months to about 2 years, from about 6 months to about 1 year, and the like. In some aspects of the methods described herein, treatment regimens may be periodically stopped and restarted according to the subject's needs.

### III. Packaged Sterile Pharmaceutical Compositions

A composition of the present disclosure may be packaged in sterile packages to facilitate administration to a patient. In some aspects the current disclosure encompasses sterile ready-to-use pharmaceutical formulations comprising the compositions provided herein packaged into user friendly products. In some aspects the package may be a single dose package. In some aspects the package maybe a multiple dose package. In some aspects the package maybe selected from a group including but not limited to a dropper bottle, bottles, squeezer dispensers, spray bottle, syringe, an ointment tube, capsule, applicator, a single use ointment pellet, single use capsules, or an implant. In some aspects the packaging may be made of glass, plastics like Low-Density Polyethylene (LDP), Polypropylene (PP), High-Density Polyethylene (HDPE), polymer or metal and any combination thereof. The packaging may be single or multilayered. In some desired aspects the packaging is leak resistant and free of undesirable contaminants. The packaging may further comprise closure mechanisms like caps, seals, films, and any mechanism known in the art.

In some aspects the packaging is designed to keep the formulation sterile till opened. In some aspects the contents comply with the sterility requirements of the standard Pharmacopoeias like United States Pharmacopoeias (USP). Sterilization may be achieved by suitable techniques such as filtration sterilization, radiation sterilization and the like.

In some aspects the packaging may be rigid or flexible in nature. The volume capacity of each unit of packaging may vary from about 10 μL to about 100 mL.

### EXAMPLES

### Example 1

A pharmaceutically-acceptable formulations were prepared as described below. The following products were used in the amounts and concentrations specified:

### Exemplary Formulations I

i. A pharmaceutically-acceptable formulation was prepared as described below. The following products were used in the amounts and concentrations specified:
ii. about 0.010 g of atropine sulfate monohydrate;
iii. about 0.20 g of poloxamer 407;
iv. about 1.00 g of polyvinylpyrrolidone (povidone k30);
v. about 0.10 g of EDTA disodium;
vi. about 0.500 g of sodium chloride;
vii. about 0.500 g of glycerin;
viii. about 100 ml of water for injection.
ix. The poloxamer 407 and polyvinylpyrrolidone (povidone K30) were added to 90% of the final volume of water for injection, then mixed until completely dissolved. The atropine sulfate monohydrate, EDTA disodium, sodium chloride and glycerin were then added one by one until completely dissolved.
x. The pH was measured and adjusted to between 5.0 to 6.0 with sodium hydroxide and/or hydrochloric acid.
xi. The solution was brought to final volume and aseptically filtered using a sterilizing 0.2 micron polyethersulfone (PES) filter.
xii. LDPE ophthalmic bottles were used with a unique two-way valve tip cap that allow for multiple dosing without the use of preservatives.

### Exemplary Formulations II

xiii. A pharmaceutically-acceptable formulation was prepared as described below. The following products were used in the amounts and concentrations specified:
xiv. about 0.010 g of atropine sulfate monohydrate;
xv. about 0.10 g of chitosan hydrochloride;
xvi. about 1.00 g of polyvinylpyrrolidone (povidone k30);
xvii. about 0.10 g of EDTA disodium;
xviii. about 0.500 g of sodium chloride;
xix. about 0.500 g of glycerin;
xx. about 100 ml of water for injection.
xxi. The chitosan hydrochloride and poloxamer 407 were added to 90% of the final volume of water for injection, then mixed until completely dissolved. The atropine sulfate monohydrate, EDTA disodium, sodium chloride and glycerin were then added one by one until completely dissolved.
xxii. The pH was measured and adjusted to between 5.0 to 6.0 with sodium hydroxide and/or hydrochloric acid.
xxiii. The solution was brought to final volume and aseptically filtered using a sterilizing 0.2 micron polyethersulfone (PES) filter.
xxiv. LDPE ophthalmic bottles were used with a unique two-way valve tip cap that allow for multiple dosing without the use of preservatives.

### Example 2

The pharmaceutically acceptable formulation II was prepared as provided in Example 1, comprising 0.01% Atropine Sulfate. The stability of atropine sulfate monohydrate and levels of impurities (specifically tropic acid) was assayed over a period of 6 months at a temperature of 25° C. (±2) and 60% (±5%) relative humidity. The assay results are provided in Table 1:

TABLE 1
Stability assay for formulation comprising Atropine sulfate

| | | Atropine Sulfate Monohydrate | | | | | | |
|---|---|---|---|---|---|---|---|---|
| Date | Time | Label | % Label | Amount | Acceptance | | Tropic Acid | |
| Tested | Point | Claim | Claim | Found | Criteria | Pass/Fail | Amount Found | pH |
| Apr. 14, 2021 | Day 0 | 0.01% | 106.20% | 0.010620% | 90-110% | Pass | Below Detectable | 5.74 |
| | | | | | | | Limits (LOD | |
| | | | | | | | 0.00022 mg/mL) | |
| Jun. 13, 2021 | Day 60 | 0.01% | 108.50% | 0.010800% | 90-110% | Pass | 0.0003 mg/mL | N/A |
| Mar. 9, 2021 | Day 120 | 0.01% | 102.90% | 0.010290% | 90-110% | Pass | 0.00085 mg/mL | N/A |
| Nov. 10, 2021 | Day 180 | 0.01% | 99.50% | 0.009950% | 90-110% | Pass | 0.00195 mg/mL | 5.72 |

Results suggest that the formulation was within clinically acceptable levels of active ingredient and impurities for at least up to 6 months on storage.

1. A sterile pharmaceutical composition for contacting an ocular surface of a mammal, the sterile pharmaceutical composition comprising:
a pharmaceutical agent,
a cationic polymer,
a non-ionic polymer, and
a pharmaceutical carrier,
wherein the duration of the clinical effect stemming from the sterile pharmaceutical composition is greater than 6 hours.

2. (canceled)

3. (canceled)

4. The sterile pharmaceutical composition of claim 1, further comprising one or more additional non-ionic polymers.

5. The sterile pharmaceutical composition of claim 1, further comprising an anionic polymer.

6. (canceled)

7. The sterile pharmaceutical composition of claim 5, wherein the anionic polymer has a concentration of about 0.2% w/v to about 2.0% w/v.

8. The sterile pharmaceutical composition of claim 1, wherein the cationic polymer has a concentration of about 0.01% w/v to about 2.0% w/v.

9. The sterile pharmaceutical composition of claim 1, wherein the non-ionic polymer has a concentration of about 0.1% w/v to about 4.0% w/v.

10. The sterile pharmaceutical composition of claim 1, wherein the pharmaceutical carrier comprises one or more pharmaceutically acceptable buffers.

11. (canceled)

12. The sterile pharmaceutical composition of claim 1, wherein the pharmaceutical carrier comprises one or more tonicity adjusting agents.

13. (canceled)

14. The sterile pharmaceutical composition of claim 1, wherein the pharmaceutical carrier comprises one or more stabilizers.

15. (canceled)

16. The sterile pharmaceutical composition of claim 1, wherein the pharmaceutical carrier comprises one or more pH adjusting agents.

17. The sterile pharmaceutical composition of claim 1, wherein the pharmaceutical carrier comprises water.

18. The sterile pharmaceutical composition of claim 1, wherein the pharmaceutical composition does not comprise a preservative.

19. The sterile pharmaceutical composition of claim 1, wherein the pharmaceutical composition has a viscosity of about 1 cP to about 20 cP.

20. The sterile pharmaceutical composition of claim 1, wherein the pharmaceutical composition has a pH of about 5.0 to about 8.0.

21. (canceled)

22. The sterile pharmaceutical composition of claim 1, wherein the pharmaceutical agent comprises a cholinesterase inhibitor selected from the group consisting of any one of physostigmine, neostigmine, pyridostigmine, ambenonium, demecarium bromide, rivastigmine, galantamine, caffeine, rosmarinic acid, alpha-pinene, donepezil, tacrine, edrophonium, huperzine A, ladostigil, ungeremine, lactucopicrin, echothiophate, diisopropyl fluorophosphatesneostigmine, echothiophate iodide (also known as phospholine iodide), aceclidine, ambenonium, demecarium, rivastigmine, galantamine, acotiamide, diisopropyl fluorophsphate, cadusafos, chlorpyrifos, cyclosarin, dichlorvos, dimethoate, metrifonate, and any combination or pharmaceutically acceptable salts thereof.

23. The sterile pharmaceutical composition of claim 22, wherein the cholinesterase inhibitor has a concentration of about 0.001% w/v to about 0.1% w/v.

24. (canceled)

25. The sterile pharmaceutical composition of claim 1, wherein the pharmaceutical agent comprises a muscarinic receptor agonist selected from the group consisting of pilocarpine, choline, acetylcholine, nicotine, methacholine, carbachol, cevimeline, CI-1017, bethanechol, milameline, muscarine, oxotremorine, sabcomeline, talsaclidine, tazomeline, vedaclidine, VU0152100, VU0238429, xenomeline, AF102B, AF150(S), AF267B, aceclidine, arecoline, pilocarpine, cevimeline, and any combination or pharmaceutically acceptable salts thereof.

26. The sterile pharmaceutical composition of claim 25, wherein the muscarinic agonist has a concentration of about 0.001% w/v to about 1% w/v.

27. (canceled)

28. The sterile pharmaceutical composition of claim 1, wherein the pharmaceutical agent comprises a muscarinic receptor antagonist selected from the group consisting of atropine, scopolamine, glycopyrrolate, ipratropium bromide and any combination thereof or any pharmaceutically acceptable salt thereof.

29. The sterile pharmaceutical composition of claim 28, wherein the muscarinic receptor antagonist has a concentration of about 0.001% w/v to about 3% w/v.

30. The pharmaceutical composition of claim 29, wherein the pharmaceutical composition has a tropic acid concentration of less than 0.01 mg/ml after 180 days of storage in a sealed container at a temperature of 25° C. (±2) and 60% (±5%) relative humidity.

31. The sterile pharmaceutical composition of claim 1, wherein the pharmaceutical composition is stable for at least 60 days, 120 days, or 180 days of storage in a sealed container at a temperature of 25° C. (±2) and 60% (±5%) relative humidity.

32. (canceled)

33. (canceled)

34. (canceled)

35. The sterile pharmaceutical composition of claim 1, wherein the cationic polymer comprises chitosan.

36. The sterile pharmaceutical composition of claim 1, wherein the non-ionic polymer is selected from the group consisting of polyvinyl pyrrolidone (Povidine K30), a poly (oxyethylene-co-oxypropylene) block copolymer (Poloxamer 407), or a combination thereof.

37. (canceled)

38. The sterile pharmaceutical composition of claim 1, wherein the pharmaceutical agent comprises an alpha-adrenergic agonist selected from the group consisting of phenoxybenzamine, phentolamine, tolazoline, trazodone, alfuzosin, dapiprazole, thymoxamine, doxazosin, prazosin, tamsulosin, bunezosin, terazosin, trimazosin, silodosin, atipamezole, idazoxan, mirtazapine, yohimbine, carvedilol, labetalol, urapidil, abanoquil, adimolol, ajmalicine, amosulalol, arotinolol, atiprosin, benoxathian, buflomedil, bunazosin, carvedilol, CI-926, corynanthine, DL-017, domesticine, eugenodilol, fenspiride, GYM-12743, GYKI-16084, indoramin, ketanserin, L-765314, mephendioxan, metazosin, monatepil, naftopidil, nantenine, neldazosin, nicergoline, niguldipine, pelanserin, phendioxan, piperoxan, quinazosin, ritanserin, RS-97078, SGB-1534, SL-890591, spiperone, talipexole, tibalosin, tiodazosin, tipentosin, tolazoline, upidosin, zolertine, a pharmaceutically acceptable salt thereof, and combinations thereof, and any combination or pharmaceutically-acceptable salts thereof.

39-95. (canceled)
